(19) United States
(12) Patent Application Publication
(10) Pub. No.: US 2022/0287610 A1
(43) Pub. Date: Sep. 15, 2022

(54) SENSORY DEVICE TO PATCH INTERFACE

(71) Applicant: Preventice Solutions, Inc., Rochester, MN (US)

(72) Inventors: Brian Walter Kronstedt, Edina, MN (US); Calvin Hedberg, Farmington, MN (US)

(21) Appl. No.: 17/680,542

(22) Filed: Feb. 25, 2022

Related U.S. Application Data
(60) Provisional application No. 63/159,307, filed on Mar. 10, 2021.

(51) Int. Cl. A61B 5/274; A61B 5/308; A61B 5/257; A61B 5/00

(52) U.S. Cl. CPC A61B 5/274; A61B 5/308; A61B 5/257; A61B 5/7405

(57) ABSTRACT

A patch includes a pad with a back surface for attaching to a patient's body and with a front surface that is on an opposite side of the back surface. The patch includes a shoe coupled to the front surface and defining an internal cavity shaped to receive a monitor. The shoe includes or defines a sensory feedback feature configured to engage with the monitor. An electrical connector is positioned within the internal cavity and is configured to mechanically and electrically couple to the monitor.

### RELATED APPLICATIONS

This application claims priority to Provisional Application No. 63/159,307, filed Mar. 10, 2021, which is herein incorporated by reference in its entirety.

### SUMMARY

Cardiac monitoring patches can include a disposable, single-use patch and a reusable monitor. The patch may be positioned a patient's body (e.g., on the patient's chest), and the monitor may then be engaged to the patch to monitor the patient's heart.

Certain instances of the present disclosure describe various features that provide sensory feedback, (e.g., tactile and/or audible feedback), when a monitor has properly engaged with a patch. This feedback reduces the risk that the monitor will be improperly or insufficiently engaged, which reduces the risk of the monitor unintentionally disengaging or sliding out of the patch.

### BRIEF DESCRIPTION OF THE DRAWINGS

The appended drawings illustrate only exemplary embodiments and are therefore not to be considered limiting of its scope.

FIG. 1 illustrates a top-down view of an example patch, in accordance with certain instances of the present disclosure.

FIG. 2 illustrates a top-down view of an example monitor engaging with the patch of FIG. 1, in accordance with certain instances of the present disclosure.

FIGS. 3A and 3B illustrate an example shoe, in accordance with certain instances of the present disclosure.

FIG. 3C illustrates an example monitor, in accordance with certain instances of the present disclosure.

FIGS. 3D and 3E illustrate the shoe of FIGS. 3A and 3B and the monitor of FIG. 3C, in accordance with certain instances of the present disclosure.

FIG. 4A illustrates an example shoe, in accordance with certain instances of the present disclosure.

FIGS. 4B and 4C illustrate an example monitor, in accordance with certain instances of the present disclosure.

FIG. 5A illustrates an example shoe, in accordance with certain instances of the present disclosure.

FIG. 5B illustrates an example monitor, in accordance with certain instances of the present disclosure.

FIGS. 5C and 5D illustrate an example universal serial bus (USB) port in the monitor of FIG. 5B, in accordance with certain instances of the present disclosure.

FIGS. 6A and 6B illustrate an example shoe, in accordance with certain instances of the present disclosure.

FIGS. 6C and 6D illustrate an example monitor, in accordance with certain instances of the present disclosure.

FIGS. 6E and 6F illustrate the shoe of FIGS. 6A and 6B and the monitor of FIGS. 6C and 6D, in accordance with certain instances of the present disclosure.

FIG. 7A illustrates an example shoe, in accordance with certain instances of the present disclosure.

FIGS. 7B and 7C illustrate an example USB connector body in the shoe of FIG. 7A, in accordance with certain instances of the present disclosure.

FIG. 8 illustrates an example shoe, in accordance with certain instances of the present disclosure.

To facilitate understanding, identical reference numerals have been used, where possible, to designate identical elements that are common to the figures. It is contemplated that elements and features of one embodiment may be beneficially incorporated in other embodiments without further recitation.

### DETAILED DESCRIPTION

Cardiac monitoring patches may use a disposable, single-use patch that is releasably coupled to a reusable monitor. One challenge with such patches is providing positive feedback to the user to help ensure a proper connection has been made between the patch and module. Without positive feedback, a user may question if a proper connection has been made between the patch and monitor or the user may be unaware of an insufficient connection. The lack of confidence that the monitor is fully engaged with the patch is especially true when the monitor is being connected while the patch is already on the patient's body. As a result of insufficient connections, the monitor may produce faulty readings or may disengage with the patch during use.

Certain instances of the present disclosure are accordingly directed to various features on the disposable patch, the reusable monitor, or both to provide sensory feedback (e.g., tactile or audible feedback) when proper engagement is achieved. This feedback alerts a user that proper connection has been made between the two units. This feedback mechanism serves not only to confirm proper connection but may also serve to lock and hold the monitor to the patch until it is intentionally released by the user.

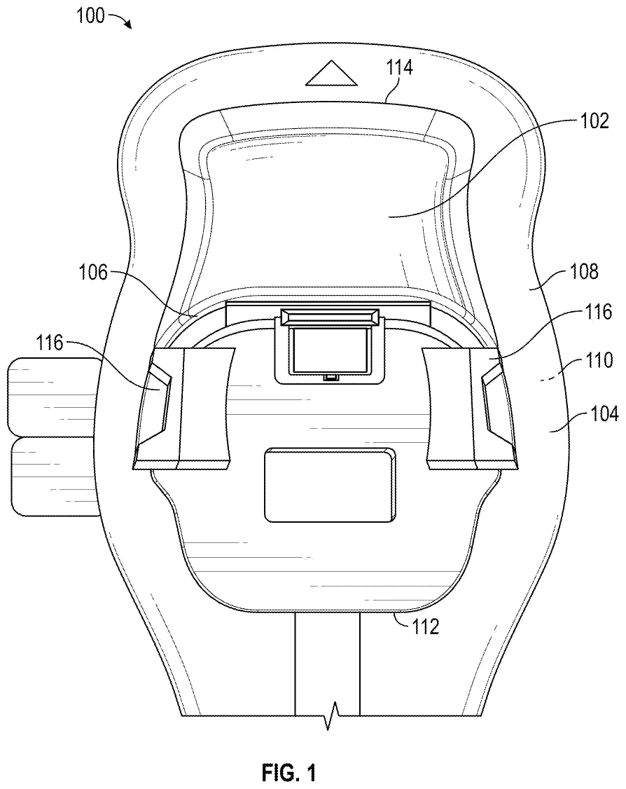

FIG. 1 illustrates a top-down view of an example patch 100. As seen in FIG. 1, the patch 100 includes a shoe 102, a pad 104, and wings 116. The shoe 102 is positioned above the pad 104 and is coupled to a top surface 108 of the pad 104. A bottom surface 110 of the pad 104 opposite the top surface 108 of the pad 104 adheres to a patient's body (e.g., on the patient's chest). The wings 116 are positioned on the left and right sides of the shoe 102 between the shoe 102 and a rear surface 112 of the shoe 102. The shoe 102 and the wings 116 may be made from a material such as plastic or metal.

The shoe 102 defines a cavity 106 that opens towards a rear surface 112 of the shoe 102. A monitor may engage the patch 100 by sliding into the cavity 106 towards a front surface 114 of the shoe 102 and coupling to a USB connector within the cavity 106. The monitor may then monitor the heartbeat of the patient (e.g., via electrodes on the monitor).

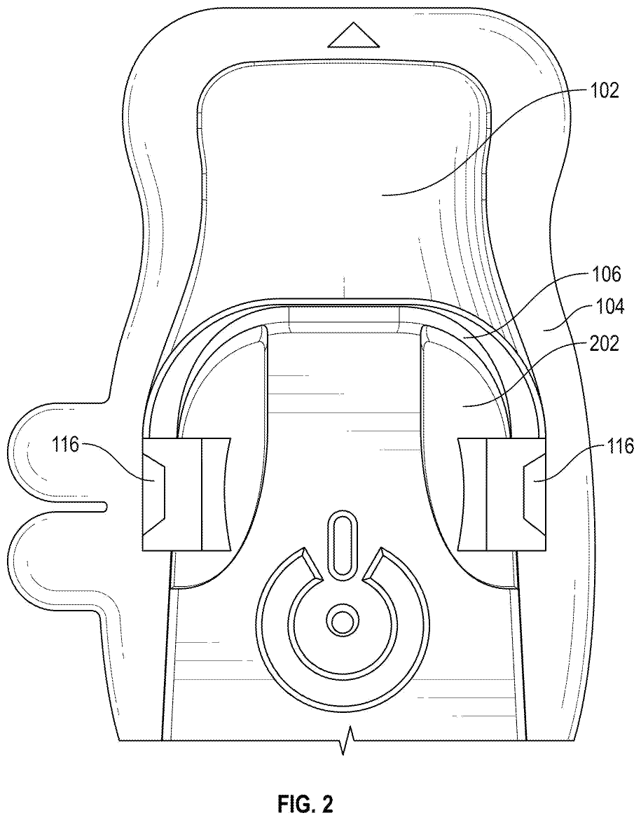

FIG. 2 illustrates a top-down view of an example monitor 202 engaging with the patch 100 of FIG. 1. As seen in FIG. 2, the monitor 202 slides into the cavity 106 of the shoe 102 to engage with the patch 100.

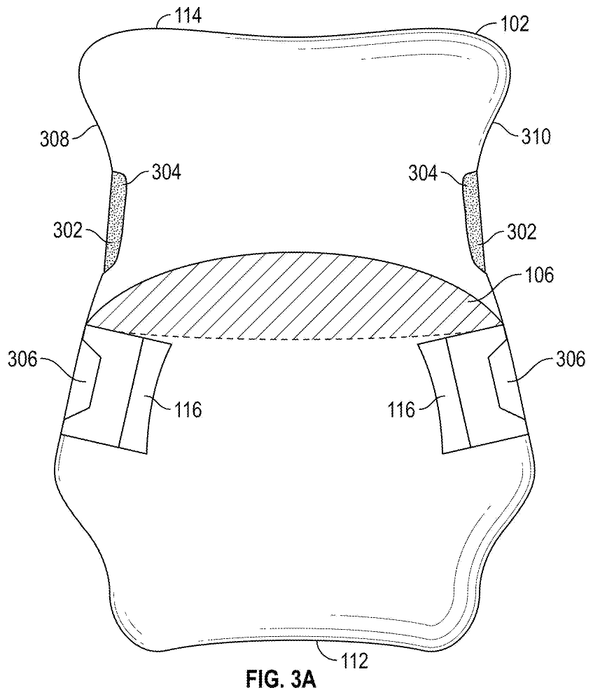

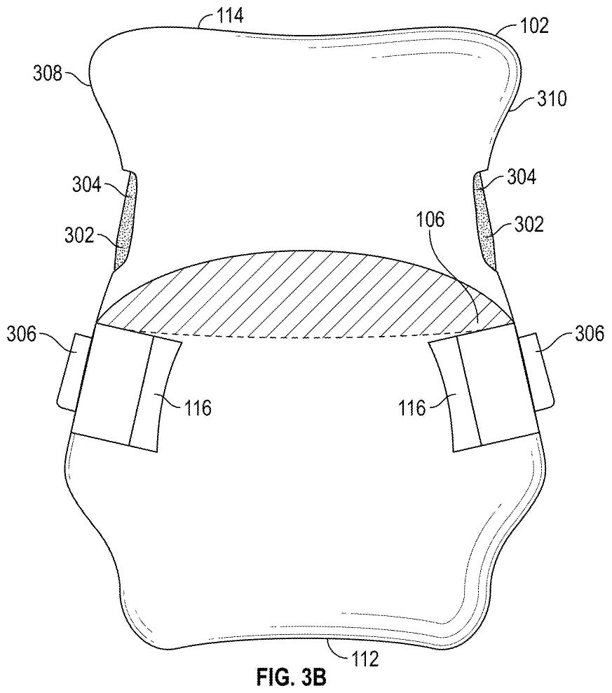

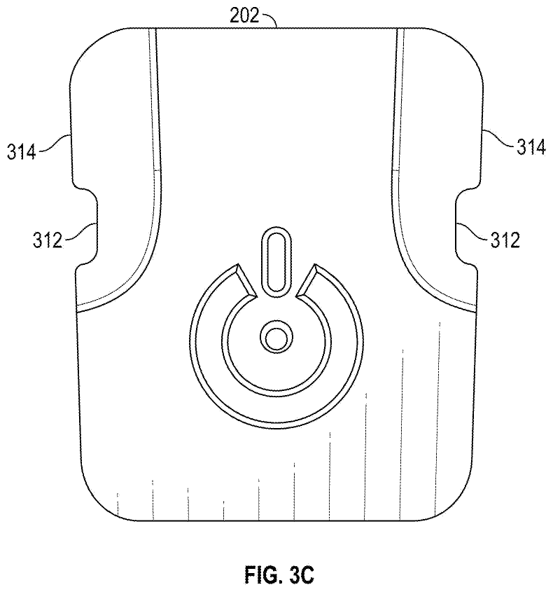

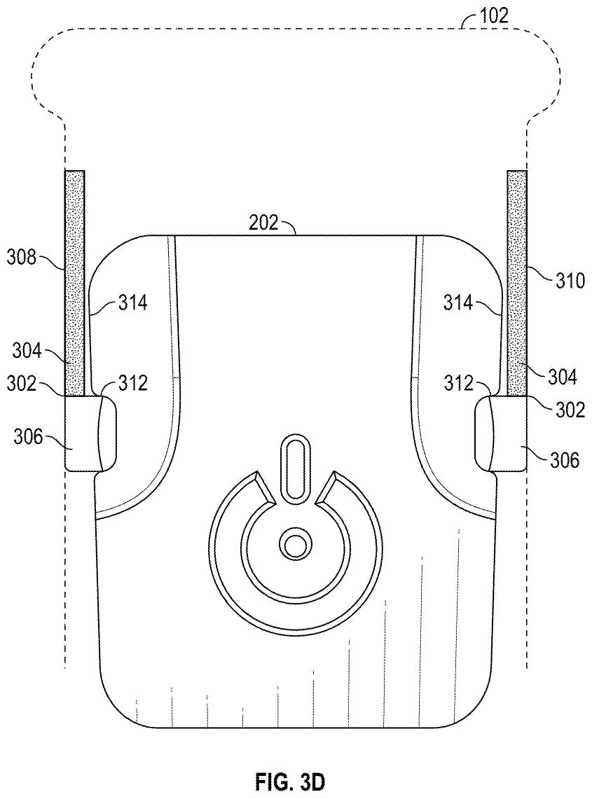

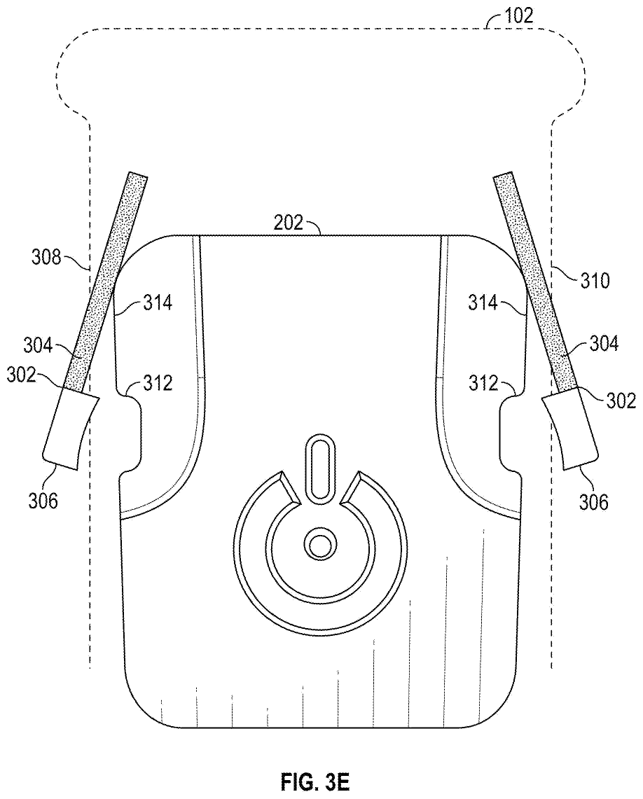

The shoe 102, the monitor 202, and/or the wings 116 can have one or more features that provide feedback indicating that the monitor 202 is properly engaged with the shoe 102. Example features are described in more detail using FIGS. 3A-3E, 4A-4C, 5A-5D, 6A-6F, 7A-7C, and 8.

FIGS. 3A and 3B illustrate top-down views of an example shoe 102. As seen in FIGS. 3A and 3B, the shoe 102 includes one or more hinges 302 positioned on a side of the shoe 102 and the wings 116. For example, the shoe 102 may include a hinge 302 positioned on a left side 308 of the shoe 102 and a hinge 302 positioned on a right side 310 of the shoe 102.

Each hinge 302 includes a first portion 304 and a second portion 306. The first portions 304 are positioned on a side 308 or 310 of the shoe 102 between the cavity 106 and the front surface 114 of the shoe 102. The second portions 306 are positioned on the wings 116. As seen in FIG. 3B, the first portions 304 may be pressed by a user to move the first portions 304 into the shoe 102. Correspondingly, the second portions 306 lift out of the wings 116.

FIG. 3C illustrates a top-down view of an example monitor 202. As seen in FIG. 3C, the monitor 202 includes slots 312 formed on the sides 314 of the monitor 202. These slots 312 engage with the second portions 306 of the hinges 302 to provide sensory feedback when the monitor 202 is properly engaged with the shoe 102.

FIGS. 3D and 3E illustrate a top-down view of the shoe 102 of FIGS. 3A and 3B, and the monitor 202 of FIG. 3C. As seen in FIG. 3D, the monitor 202 includes slots 312 that align with the second portions 306 of the hinges 302 when the monitor 202 is properly engaged with the shoe 102. The second portions 306 engage with the slots 312 to secure the monitor 202. Additionally, when the second portions 306 engage with the slots 312, sensory feedback (e.g., tactile or audible feedback) may be generated. For example, the second portions 306 may produce an audible sound (e.g., a “click”) when the second portions 306 engage with the slots 312 (e.g., due to the second portions 306 snapping into place within the slots 312). In some embodiments, the second portions 306 engage the slots 312 simultaneously to provide the sensory feedback. As seen in FIG. 3E, when a user presses on the first portions 304, the second portions 306 disengage with the slots 312 on the monitor 202. As a result, the monitor 202 disengages from the shoe 102 and may be removed. In the example of FIGS. 3D and 3E, a user would press on the first portions 304 on the left side 308 and the right side 310 of the shoe 102 simultaneously to disengage the second portions 306 from the monitor 202.

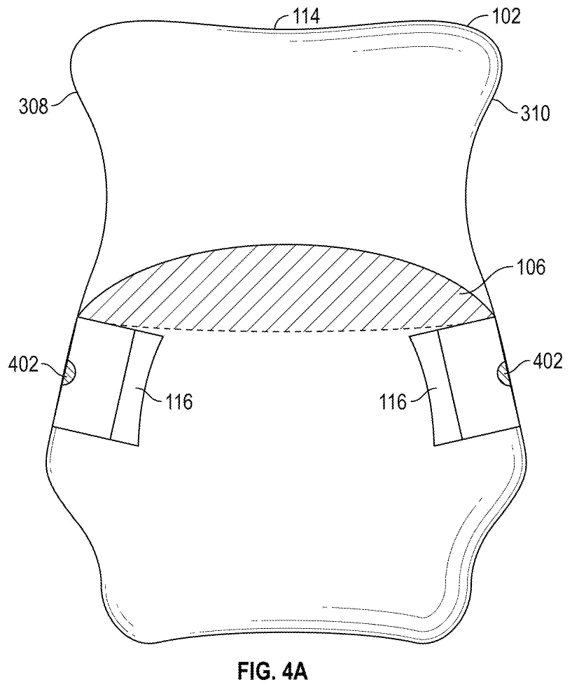

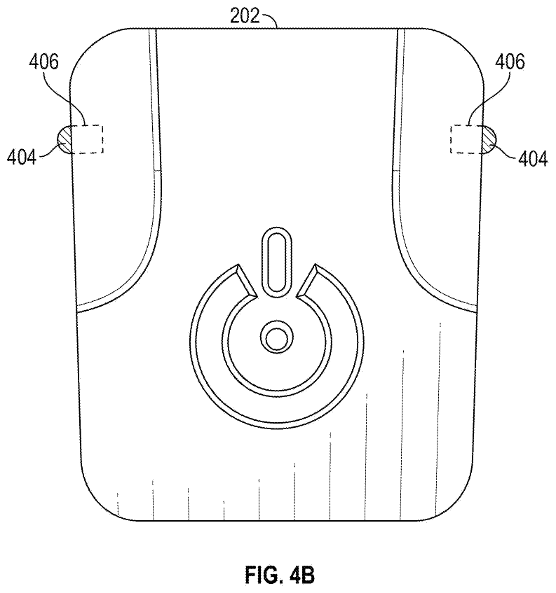

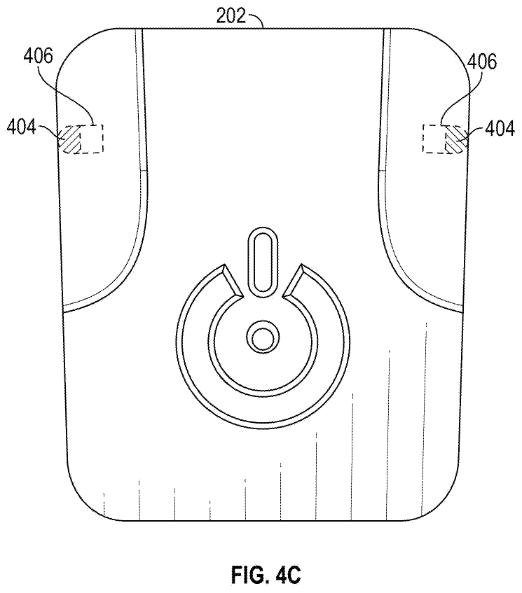

FIG. 4A illustrates a top-down view of an example shoe 102. As seen in FIG. 4A, the shoe 102 includes one or more slots 402 on a side of the wings 116. For example, each wing 116 may include a slot 402. The slots 402 may be holes or cavities defined by the wings 116. Generally, the monitor 202 is provisioned with features that register with the slots 402 when the monitor 202 is properly engaged with the shoe 102. One embodiment of such a monitor is illustrated in FIGS. 4B and 4C.

FIGS. 4B and 4C show top-down views of the monitor 202. As seen in FIGS. 4B and 4C, the monitor 202 includes buttons 404 positioned on the sides of the monitor 202. The buttons 404 are sized and located to register with the slots 402 in the wings 116 when the monitor 202 is properly engaged with the shoe 102. For example, the buttons 404 may extend into the slots 402 when the monitor 202 is properly engaged with the shoe 102. The buttons 404 may produce sensory feedback (e.g., a click) when the buttons 404 engage the slots 402.

To this end, the buttons 404 may be provisioned with compressible elements 406. In one embodiment, the compressible elements 406 are springs, so that the buttons 404 are spring-loaded. In other embodiments, the compressible elements 406 are foam or rubber. In operation, and as seen in FIG. 4C, the buttons 404 may be urged inward (e.g., toward one another and into the monitor 202) by application of a lateral force, thus compressing the compressible elements 406. In the absence of such a lateral force, the buttons 404 return to their default states, as shown in FIG. 4B. For example, when a user or a side of the wings 116 presses on the buttons 404, the buttons 404 may move inward. When the user stops pushing on the buttons or when the buttons 404 register with the slots 402 in the wings 116, the compressible elements 406 may push the buttons 404 outwards again. The desired tactile or audible feedback may be produced, for example, by the compressible element itself, or by contact between the buttons 404 and the respective surfaces of the slots 402. Additionally, when the buttons 404 are engaged with the slots 402 in the wings 116, the buttons 404 and the slots 402 may secure the monitor 202 within the shoe 102 and prevent the monitor 202 from unintentionally disengaging the shoe 102. For example, the buttons 404 and the slots 402 may prevent the monitor 202 from disengaging the shoe 102 unless sufficient lateral force is applied to the buttons 404.

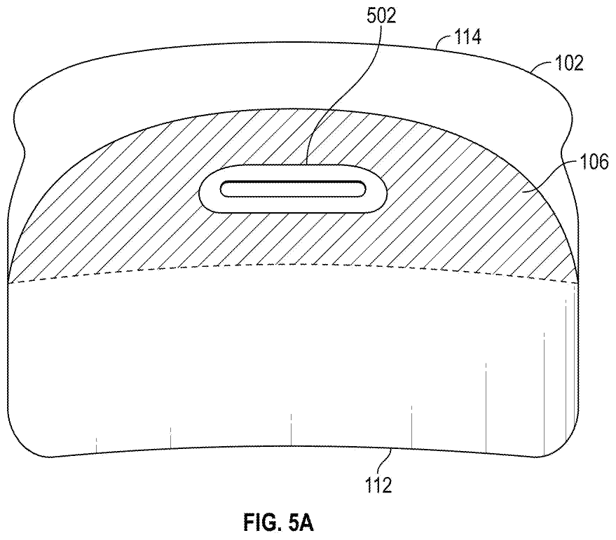

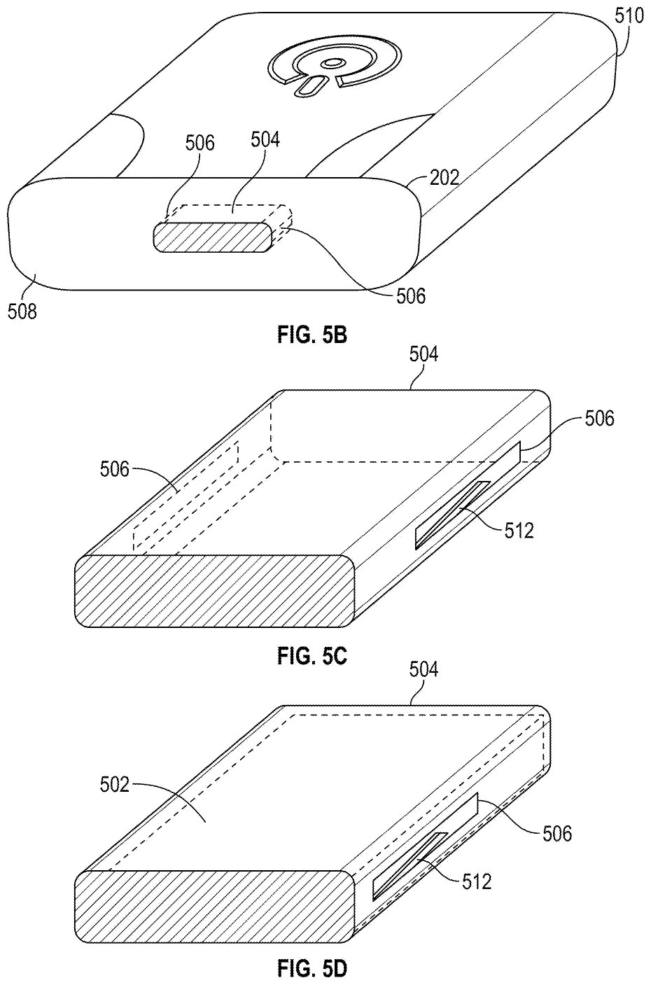

FIG. 5A illustrates an example shoe 102. FIG. 5A illustrates a view of the shoe 102 from a rear surface 112 of the shoe 102 towards a front surface 114 of the shoe 102 and into the cavity 106. As seen in FIG. 5A, a USB connector 502 (e.g., a male micro-USB connector) is positioned within the cavity 106 near the front surface 114. Generally, a monitor connects to the shoe 102 by connecting with the USB connector 502. Data and information are then communicated to the monitor 202 through the USB connector 502. For example, detected signals representing a patient's heartbeat are communicated through the USB connector 502.

FIG. 5B illustrates an example monitor 202. FIG. 5B illustrates a view of the monitor 202 from a front surface 508 of the monitor 202 towards a rear surface 510 of the monitor 202. The front surface 508 of the monitor 202 includes a USB port 504 (e.g., a female micro-USB connector and its corridor in the monitor 202) that engages the USB connector 502 of the shoe 102 to form a USB connection. For example, a user may apply a force to press the USB connector 502 towards the USB port 504. The USB connector 502 may engage the USB port 504 by fitting into the USB port 504 to form the USB connection. Data signals (e.g., data representing a patient's heartbeat) may then be communicated between the shoe 102 and the monitor 202 using this USB connection.

Rails 506 may be positioned on the sides of the USB port 504. These rails 506 may allow the USB port 504 to move inwards when the USB connector 502 is pressed against the USB port 504 to form the USB connection. FIG. 5C illustrates the USB port 504 and the rails 506. As seen in FIG. 5C, the rails 506 are positioned on the sides of the USB port 504. The rails 506 allow the USB port 504 to move along the rails 506 such that the USB port 504 moves towards the inside of the monitor 202 when the USB connector 502 and the USB port 504 are pressed together to connect the USB connector 502 and the USB port 504. The rails 506 include a latch 512. When the USB port 504 is pressed inwards, the USB port 504 moves inwards along the rails 506, The latches 512 move to provide tactile or audible feedback (e.g., a click) when the USB port 504 has moved inwards a certain distance. FIG. 5D illustrates the USB connector 502 engaged with the USB port 504. As seen in FIG. 5D, the USB port 504 has moved along the rails 506 because the USB port 504 has experienced enough inward force to have engaged properly with the USB connector 502. The latches 512 may have provided the feedback that indicates that the monitor 202 has properly engaged the shoe 102.

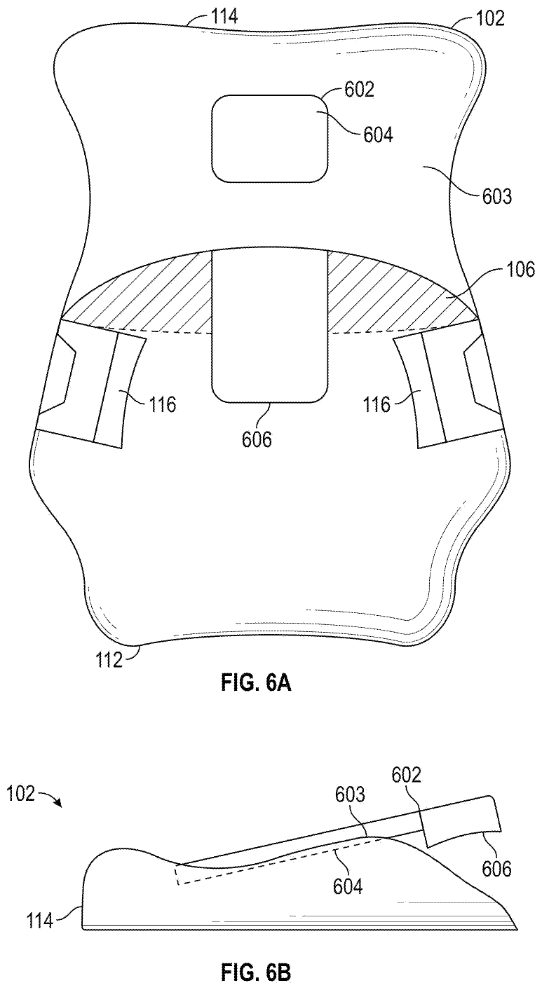

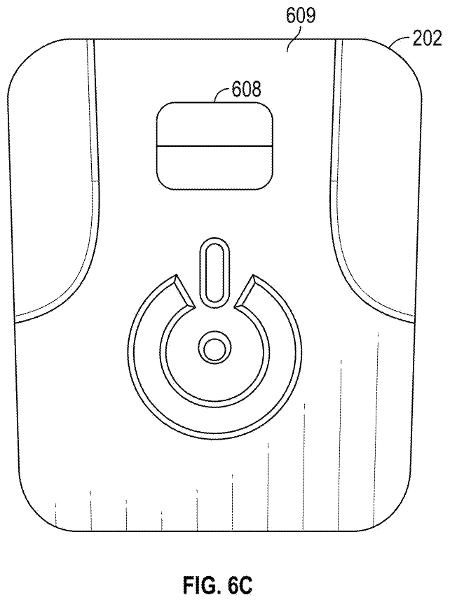

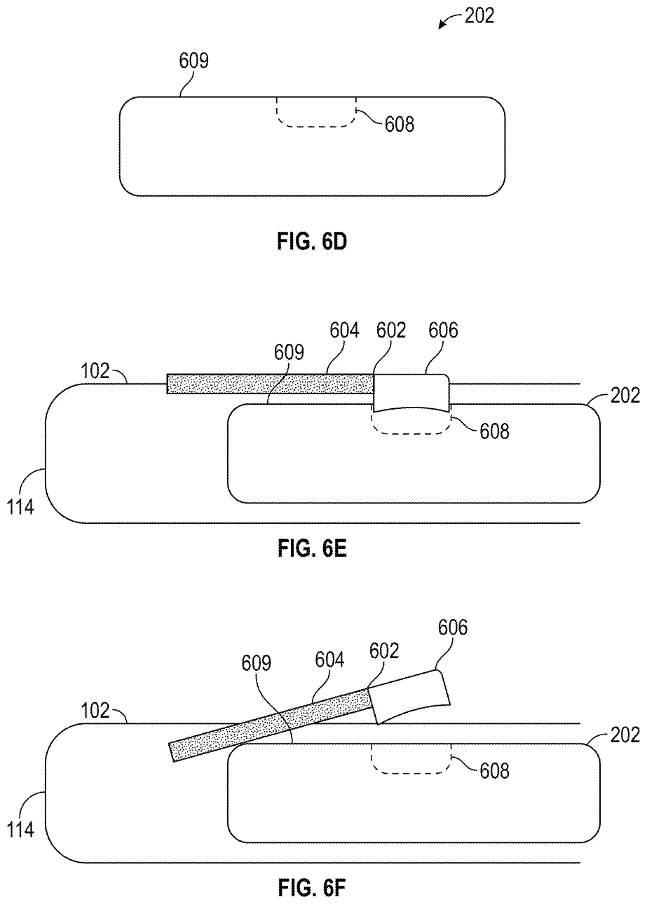

FIG. 6A illustrates a top-down view of an example shoe 102, and FIG. 6B illustrates a side view of the shoe 102. As seen in FIG. 6A, the shoe 102 includes a hinge 602 on a top surface 603 of the shoe 102. Similar to the hinge 302, the hinge 602 includes a first portion 604 and a second portion 606. The second portion 606 extends from the first portion 604 towards the rear surface 112 of the shoe 102 and past the cavity 106. As seen in FIG. 6B, when the first portion 604 is pressed downwards into the shoe 102, the second portion 606 is lifted up.

FIG. 6C illustrates a top-down view of an example monitor 202, and FIG. 6D illustrates a side view of the monitor 202. As seen in FIGS. 6C and 6D, the monitor 202 includes a slot 608 positioned on a top surface 609 of the monitor 202. FIGS. 6E and 6F illustrate side views of the shoe 102 of FIGS. 6A and 6B and the monitor 202 of FIGS. 6C and 6D. As seen in FIG. 6E, the hinge 602 engages with the slot 608 when the monitor 202 is properly positioned within the shoe 102. Specifically, the second portion 606 engages with the slot 608 and prevents the monitor 202 from unintentionally disengaging from the shoe 102. Moreover, when the second portion 606 engages with the slot 608, sensory feedback (e.g., tactile or audible feedback) may be produced. As seen in FIG. 6F, a user may press on the first portion 604 of the hinge 602 into the shoe 102 towards the monitor 202. Correspondingly, the second portion 606 lifts up and disengages from the slot 608. As a result, the monitor 202 disengages from the shoe 102 and may be removed from the shoe 102.

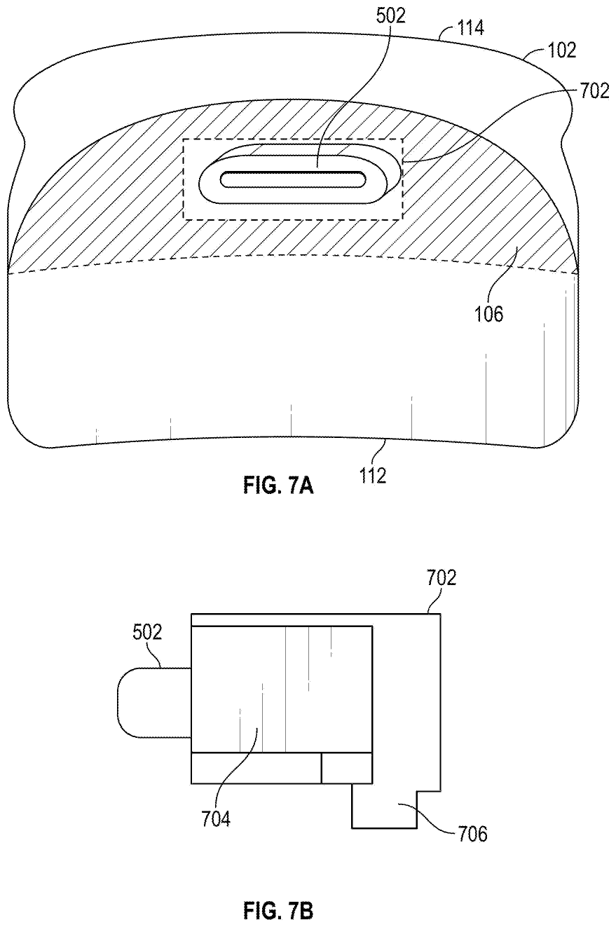

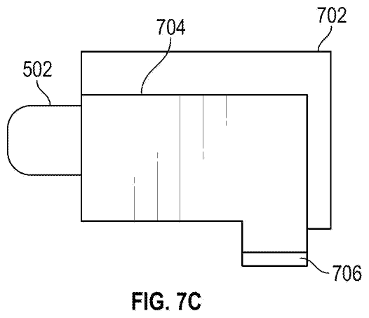

FIG. 7A illustrates a view of an example shoe 102 from a rear surface 112 of the shoe 102 towards a front surface 114 of the shoe 102 and into the cavity 106. As seen in FIG. 7A, a USB connector (e.g., a male micro-USB connector) 502 is positioned within the cavity 106. Generally, a monitor 202 connects to the shoe 102 by connecting with the USB connector 502. Data and information are then communicated to the monitor 202 through the USB connector 502. For example, detected signals representing a patient's heartbeat are communicated through the USB connector 502. Additionally, as seen in FIG. 7A, the shoe 102 includes an internal recess 702 behind the USB connector 502. When sufficient force is exerted on the USB connector 502 (e.g., sufficient force to connect a USB connector on the monitor 202 to a USB port on the shoe 102), the USB connector 502 may move into this recess. As the USB connector 502 moves into the recess 702, the USB connector 502 and the recess 702 provide tactile or audible feedback (e.g., a click) that alerts a user that the monitor 202 has properly engaged the shoe 102.

FIGS. 7B and 7C illustrate an example USB connector 502 in the shoe 102 of FIG. 7A. As seen in FIG. 7B, the recess 702 is positioned behind the USB connector 502. The USB connector 502 includes a body 704 that moves backwards into the recess 702 when sufficient force (e.g., 8 pounds) is exerted on the USB connector 502. The recess 702 includes a slot 706 at a bottom portion of the recess 702. As seen in FIG. 7C, when the body 704 moves into the recess 702, a portion of the body 704 slides down and into the slot 706. This engagement with the slot 706 provides tactile and audio feedback. In particular embodiments, it may not be possible pull the body 704 out of the slot 706 or the recess 702 once the portion of the body 704 slides into the slot 706. As a result, the USB connector 502 may provide the tactile or audio feedback only once, consistent with the single-use nature of the patch 100.

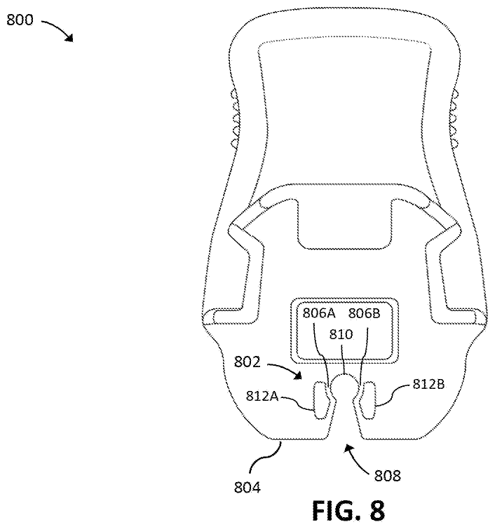

FIG. 8 illustrates a top-down view of an example shoe 800 for use with the patch 100 of FIG. 1. Similar to the shoe 102 of FIG. 1, the shoe 800 defines a cavity that opens towards the rear surface (or proximal end) and that is shaped to receive a monitor.

The shoe 800 includes a sensory feedback feature 802 at the proximal end 804 of the shoe 800. In the embodiment of FIG. 8, the sensory feedback feature 802 is defined by—and an integral portion of—the shoe 800. The sensory feedback feature 802 includes two arms 806A and 806B. The shoe 800 (e.g., via its outer edge) defines a central channel 808 that extends between the two arms 806A and 806B. The central channel 808 leads to a circular hole 810. In embodiments, the central channel 808 is a proximal portion of the sensory feedback feature 802, and the circular hole 810 is a distal portion of the sensory feedback feature 802. As shown in FIG. 8, the central channel 808 can narrow as the central channel 808 extends towards the circular hole 810. Together, the central channel 808 and the circular hole 810 form a keyhole-shaped opening.

The sensory feedback feature 802 can include two outer openings 812A and 812B. With the central channel 808, circular hole 810, and the two outer openings 812A and 812B, the sensory feedback feature 802 defines the two arms 806A and 806B.

The monitor can include a feature that—as the monitor is inserted into the shoe 800—is inserted into the sensory feedback feature 802. As the feature passes through the central channel 808, the feature can apply a force on the two arms 806A and 806B such that they bend or deform. When the feature of the monitor passes into the circular hole 810, the two arms 806A and 806B can snap back into their original shape and position which creates an audible sound (e.g., a “click”) and tactile feedback. The shape and position of the two arms 806A and 806B can be designed such that they snap back once the monitor has properly connected to the electrical connector.

In the preceding, reference is made to embodiments presented in this disclosure. However, the scope of the present disclosure is not limited to specific described embodiments. Instead, any combination of the described features and elements, whether related to different embodiments or not, is contemplated to implement and practice contemplated embodiments. Furthermore, although embodiments disclosed herein may achieve advantages over other possible solutions or over the prior art, whether or not a particular advantage is achieved by a given embodiment is not limiting of the scope of the present disclosure. Thus, the preceding aspects, features, embodiments and advantages are merely illustrative and are not considered elements or limitations of the appended claims except where explicitly recited in a claim(s).

Additionally, in the preceding, positional and directional descriptors (e.g., top, bottom, upper, lower, front, rear, side etc.) are used to describe particular example features. These descriptors are used in reference to their respective figures. For example, a user may hold an example shoe or monitor upside-down without altering which portions are considered the front and rear portions or the top and bottom portions. As another example, a user may flip or rotate an example shoe or monitor without altering which portions are considered the top and bottom portions.

We claim:

1. A patch comprising:
a pad including a back surface for attaching to a patient's body and including a front surface that is on an opposite side of the back surface;
a shoe coupled to the front surface and defining an internal cavity shaped to receive a monitor, the shoe including or defining a sensory feedback feature configured to move into place to engage with the monitor; and
an electrical connector positioned within the internal cavity and configured to mechanically and electrically couple to the monitor.

2. The patch of claim 1, wherein the sensory feedback feature is further configured to lock the monitor into position.

3. The patch of claim 1, wherein the sensory feedback feature is further configured to create audible feedback.

4. The patch of claim 3, wherein the audible feedback is a click noise.

5. The patch of claim 3, wherein audible feedback is caused by the sensory feedback feature snapping into place.

6. The patch of claim 3, wherein the sensory feedback feature is configured to create the audible feedback only once the monitor is locked into position.

7. The patch of claim 3, wherein the sensory feedback feature is configured to create the audible feedback only once the monitor connected to the electrical connector.

8. The patch of claim 3, wherein the sensory feedback feature is configured to create the audible feedback only once the monitor is properly engaged with the shoe.

9. The patch of claim 1, wherein the sensory feedback feature includes a bar that is hinged such that one end of the bar can selectively engage and disengage with the monitor.

10. The patch of claim 1, wherein the sensory feedback feature includes bendable arms.

11. The patch of claim 10, wherein the bendable arms are positioned on opposing sides of a central channel.

12. The patch of claim 10, wherein the bendable arms are integral portions of the shoe, wherein the shoe comprises plastic.

13. The patch of claim 1, wherein the sensory feedback feature is defined by the shoe and includes a keyhole-shaped opening.

14. The patch of claim 13, wherein the keyhole-shaped opening is positioned on a proximal end of the shoe.

15. The patch of claim 13, wherein the keyhole-shaped opening includes a proximal channel leading to a distal circular hole.

16. The patch of claim 1, wherein the patch is a single-use patch.

17. The of claim 1, wherein the electrical connector is a universal serial bus (USB) electrical connector.

18. An assembly comprising:
a monitor that is reusable and that includes a recess and a first electrical connector; and
a single-use patch comprising:
a shoe defining an internal cavity, wherein the monitor is positioned within the internal cavity, the shoe including or defining a sensory feedback feature that is engaged with the recess of the monitor, and
a second electrical connector positioned within the internal cavity and mechanically and electrically coupled to the first electrical connector.

19. The assembly of claim 18, wherein the sensory feedback feature is further configured to create audible feedback.

20. The assembly of claim 19, wherein audible feedback is caused by the sensory feedback feature snapping into the recess.
